(19) United States
(12) Patent Application Publication
(10) Pub. No.: US 2026/0306532 A1
(43) Pub. Date: Oct. 8, 2026

(54) METHOD FOR SYNTHESIZING ZEOLITE NANOPARTICLES IN A SALINE PHOSPHATE BUFFER AND ASSOCIATED NANOPARTICLES, SUSPENSIONS, PHARMACEUTICAL COMPOSITION AND USES

(71) Applicants: UNIVERSITÉ DE CARB NORMANDIE, Caen (FR); ECOLE NATIONALE SUPÉRIEURE D'INGÉIEURS DE CAEN, Caen (FR); CENTRE NATIONAL DE LA RECHERCHE SCIENTIFIQUE, Paris (FR)

(72) Inventors: Samuel VALABLE, Colomby-Anguerny (FR); Svetlana MINTOVA, Basly (FR); Charly HÉLAINE, Cairon (FR); Abdallah AMEDLOUS, Epron (FR)

(21) Appl. No.: 19/166,086

(22) PCT Filed: Mar. 19, 2024
(86) PCT No.: PCT/EP2024/057254

§ 371 Date: Sep. 17, 2025

(30) Foreign Application Priority Data
Mar. 28, 2023 (FR) 2302982

(51) Int. Cl. A61K 33/06; A61K 9/10; A61K 9/51; A61K 45/06; A61K 49/18; A61P 35/00; B82Y 5/00; B82Y 40/00; C01B 39/20

(52) U.S. Cl. CPC A61K 33/06; A61K 9/10; A61K 9/5192; A61K 45/06; A61K 49/1806; A61K 49/183; A61P 35/00; C01B 39/20; B82Y 5/00; B82Y 40/00; C01P 2004/03; C01P 2006/12; C01P 2006/14

(57) ABSTRACT

The present invention relates to a method for synthesising nanoparticles consisting of or comprising at least one zeolite nanocrystal according to which
a first composition/solution 1 containing an aluminium source and a source of an ion of an alkali metal M, in particular Na, is prepared and
a second composition/solution 2 comprising a silicon source and a source of an ion of an alkali metal M, in particular Na, is prepared. The solutions 1 and 2 being free of any organic structuring agent.

The two compositions/solutions 1 and 2 are mixed, and
the mixture is crystallised, The
nanoparticles thus formed are optionally separated.

According to the invention, said first composition/solution 1 and the second composition/solution 2 both consist of the source and of an alkaline phosphate buffer (PBS).

The present invention also relates to the nanoparticles obtained and compositions containing them.

### TECHNICAL FIELD

The present invention relates to a method for synthesising zeolite nanoparticles, the nanoparticles thus obtained, suspensions of these nanoparticles in water or PBS buffer as well as a pharmaceutical composition and a combination product containing these nanoparticles.

These nanoparticles may particularly be used in the medical field, in particular for the treatment of hypoxic solid tumours.

### PRIOR ART

Zeolites are a class of crystalline microporous materials. They are formed by a three-dimensional arrangement of TO₄ (T=Si, Al or other heteroatoms) bonded to one another by sharing an oxygen atom. In aluminosilicate zeolites, the presence of AlO₄⁵⁻ ions gives the framework of negative charges that must be compensated by so-called compensation cations (alkaline or alkaline-earth), such as Na⁺, K⁺, Ca²⁺, Sr²⁺ in the aim of maintaining electrical neutrality. The empty space is defined by the aluminosilicate framework formed by these three-dimensional chains. The vacant spaces of the tetrahedral crystal lattice are connected to each other and occupied by alkali ions and water molecules. Dehydration of the zeolites makes it possible to remove the water molecules present. Zeolites also include a microporous network formed of cavities, channels connecting the cavities, and pores connecting the channels or cavity to the outside. Zeolites can be natural or synthetic. There are currently more than 250 types of zeolites.

Document WO2015/101800 A describes a method for synthesising faujasite nanocrystals (FAU-type zeolites), faujasite monocrystals, and a method for synthesising a colloidal suspension of these nanocrystals. The faujasite nanocrystals have a size ranging from 5 to 400 nm, a Si/Al molar ratio ranging from 1 to 2.5, and a Si/Na molar ratio of 1.1 to 1.5. The total pore volume (total porous volume) ranges from 0.50 to 1.5 cm³/g.

Document WO 2019/030200 A1 describes FAU- and EMT-type zeolite nanocrystals for which sodium ions have been replaced by Fe or Gd ions and which may be charged with gaseous dioxygen. These nanocrystals may be used for the diagnosis and treatment of brain tumours, including glioblastoma.

The publication by Anfray et al “nanosized zeolites as a gas delivery platform in a glioblasma model” describes a suspension of FAU-type nanozeolites in water. The zeolite nanocrystals described in these documents are synthesised in water, which restricts the medical uses thereof. Indeed, over the long term and at high concentrations, these nanocrystals are likely to cause allergy-type problems.

### Technical Problem to be Solved

One aim of the present invention is therefore to propose novel zeolite nanoparticles that can be used in the diagnosis and/or treatment of certain pathologies, particularly solid brain tumours.

Another aim of the invention is to propose a novel method for synthesising such nanoparticles.

Another aim of the invention is to propose colloidal suspensions of these nanoparticles.

Another aim of the invention is to propose zeolite nanoparticles that have reduced toxicity and/or have improved oxygen adsorption and/or release capacity.

### SUMMARY OF THE INVENTION

A first aspect of the invention relates to a method for synthesising nanoparticles consisting of or comprising at least one zeolite nanocrystal according to which:
a first composition/solution 1 containing an aluminium source and a source of an ion of an alkali metal M, in particular Na, is prepared;
a second composition/solution 2 comprising a silicon source and a source of an ion of an alkali metal M, in particular Na, is prepared, said compositions/suspensions 1 and 2 being free of any organic structuring agent;
the compositions/solutions 1 and 2 are mixed and the mixture is left under stirring,
the mixture is crystallised at a temperature greater than or equal to 50° C.; and
said nanoparticles thus formed are optionally separated,

Characteristically, according to the invention, said first composition/solution 1 and said second composition/suspension 2 are each a composition/solution consisting of said source and of an aqueous buffer (PBS), which is consisting of water and 137 mM/L of NaCl, 2.7 mM/L of KCl, 10 mM/L of Na₂HPO₄, 1.76 mM/L of KH₂PO₄ and having a pH equal to 7.2 and said mixture of solutions 1 and 2 has the following molar composition (I):

x1M₂O:yAl₂O₃:10SiO₂:z PBS (I)

wherein 2.8≤x1≤8.8,

$$0.3 \leq y \leq 1.5 and$$

$$40 \leq z \leq 300 .$$

The method of the invention is simple to implement and makes it possible to obtain zeolite nanoparticles forming a stable colloidal suspension in PBS.

Preferably, solutions 1 and 2 are prepared.

According to the implementation, it is sometimes necessary to heat the mixture of PBS buffer and of other reagents to obtain a clear or limpid suspension.

As regards temperatures, the first composition/solution 1 may be prepared at room temperature. The second composition/solution is prepared, for example, at a temperature less than 30° C. and particularly equal to 25° C. The mixture of the two compositions/solutions may advantageously be performed at a temperature equal to or greater than 0° C. and equal to or less than 30° C. and particularly equal to 0° C. Subsequently, the mixture must be left under stirring at a temperature equal to or greater than 0° C. and equal to or less than 30° C. and particularly equal to 25° C. for at least 2 h and particularly 24 h. The mixture may be crystallised at a temperature greater than or equal to 50° C.

### DETAILED DESCRIPTION

The aluminium source may be selected from any aluminium source capable of providing monomeric oxyhydroxide species. For the purposes of the present invention, “any source of aluminium capable of providing monomeric oxyhydroxide species” means that this source of aluminium does not provide polymeric oxyhydroxide species of aluminium and makes it possible to dissolve all of the aluminium in the clear aqueous solution 1. In particular, the aluminium source may be selected from alumina, hydrated alumina, aluminium powder, Al, Al₂(SO₄)₃, aluminium hydroxide Al(OH)₃, sodium aluminate (NaAlO₂), potassium aluminate (KAlO₂) and kaolin clays.

The silicon source may be selected from all of the silicon sources likely to provide Si₂—Si₆ monomeric or oxyhydroxide species. Thus, the amount of larger units [SiOOH]ₙ is limited in the composition/suspension 2.

In particular, the silicon source may be selected from silica hydrogel, silica acid, colloidal silica, pyrogenic silica, tetraalkyl orthosilicates, silica hydroxides, precipitated silica, silica smoke and clays. Colloidal silica is preferred in all of the embodiments of the method of the invention.

The alkali metal M source may be selected particularly from a source of Na and a source of K. The hydroxides of these two alkali metals are preferred as alkali metal sources.

According to a particular embodiment, M=Na, said aluminium source is aluminium powder, solutions 1 and 2 are prepared and before crystallising said mixture of solutions 1 and 2, it is dehydrated under vacuum and the remaining solid fraction is subsequently crystallised at a temperature greater than or equal to 30° C. and less than or equal to 100° C. and in particular equal to 50° C. for a duration equal to or greater than 6 h and equal to or less than 30 h and particularly equal to 25 h.

According to a particular alternative embodiment, 5≤x1≤8.8, 0.3≤y≤1.5 and 40≤z≤200 and according to a more particular alternative embodiment x1=8.3, y=1.1 and z=55.6.

FAU-type zeolite nanoparticles are thus obtained. It should be noted that the method of the invention also makes it possible to directly obtain suspensions of zeolite nanoparticles in PBS.

Advantageously, the purification of said nanoparticles is performed using a membrane, particularly a dialysis membrane. The operation of this method is simple, inexpensive, usually carried out using commercially available membranes (dialysis membrane), and it also makes it possible to save water. This technique also makes it possible to retain the PBS in the nanoparticles.

A second aspect of the invention relates to a method for manufacturing a colloidal suspension of nanoparticles, according to which nanoparticles are synthesised according to the method of the invention and said synthesised nanoparticles are dispersed in optionally sterile water or in an optionally sterile aqueous buffer solution consisting of water and 137 mM/L of NaCl, 2.7 mM/L of KCl, 10 mM/L of Na₂HPO₄ and 1.76 mM/L of KH₂PO₄ and having a pH equal to 7.2. Thus, a suspension of zeolite nanoparticles is obtained in water or in PBS. In the case of a suspension in water, the zeolite nanoparticles that have been filtered beforehand are dispersed to remove the PBS.

The present invention also relates to a nanoparticle consisting of or comprising a zeolite nanocrystal and obtainable according to the method of the invention. According to the invention, said zeolite is an FAU-type aluminosilicate and it contains ions of the PBS, that is to say Na⁺, K⁺, H₂PO₄²⁻, HPO₄⁻H₃O⁺ and Cl⁻ ions and possibly water molecules. These ions are disposed on the surface of the crystal lattice and/or in the interstices of the crystal lattice and/or in the porous network of the particle. The surface of the crystal lattice may be the surface of the particle. The Na⁺ and K⁺ ions, particularly, can be found in the interstices of the crystal lattice as compensation ions. Said nanozeolite may advantageously contain at least two species of the aforementioned ions. Depending on the pH, it may contain different amounts of HPO₄²⁻, H₃O+, H₂PO₄⁻ ions. These ions, whatever they are, are located in the porous network thereof and/or on the surface thereof and/or in the gaps of the crystal lattice thereof and/or on the surface of the crystal lattice, which may correspond to the surface of the particle.

The inventors demonstrated that nanoparticles synthesised in PBS saline buffer are less cytotoxic and therefore more likely to be accepted by the organism.

PBS has the same concentration of salts as the human body; it also includes the dihydrogen phosphate/hydrogen phosphate pair which makes it possible to maintain the blood pH in the body. Without the inventors being related to this explanation, it would appear that certain ions of the PBS contained in the crystal lattice and/or in the porous network and/or on the surface of the nanoparticle can stabilise the nanoparticles and prevent them from degrading in the biological environment, thereby reducing the toxicity potential thereof. In addition, the ions of the PBS may also regulate the charge on the surface of nanoparticles, which may influence the behaviour thereof and the interaction thereof with biological cells and tissues. The nanoparticles obtained according to the method of the invention thus avoid or reduce the allergy phenomena described previously.

The nanoparticle may be a nanocrystal.

This nanocrystal may have an average diameter greater than or equal to 10 nm and less than or equal to 100 nm and particularly equal to 30 nm. According to a particular embodiment, zeolite has the following chemical composition:

|Na_{69.79}K_{6.06} (H₂O)_{211.85}|(Si_{123.09}Al_{66.59}O₃₈₄) (II).

The mole number of water is determined by thermogravimetric analysis. The number of atoms Na, K, Si and Al is determined by ICP, and the mole number of oxygen is determined from the mole number of metals using the stoichiometric formula.

Regardless of the embodiment, the nanoparticle of the invention may have a total pore volume greater than or equal to 0.4 cm³/g and less than or equal to 1.5 cm³/g and/or a specific surface area S_{BET} greater than or equal to 200 m²/g and less than or equal to 1,000 m²/g.

According to a particular alternative embodiment, said nanoparticle has a total porous volume (cavity+pores+channels) greater than or equal to 0.6 cm³/g and less than or equal to 1.3 cm³/g to and particularly equal to 0.93 cm³/g and/or a specific surface area S_{BET} greater than or equal to 300 m²/g and less than or equal to 800 m²/g and particularly equal to 600 or 428 m²/g. This nanocrystal may have the chemical formula (II)

The porous network of the zeolite of the invention extends along the three directions of space

According to a fourth aspect of the invention, it relates to a nanoparticle as aforementioned that further contains, in the cavities of the crystal lattice thereof, cations of a metal selected from Fe, Gd, Cu and Ce and in particular in an amount greater than or equal to 0.2% by weight and less than or equal to 5% by weight and/or a gas contained in the porous volume thereof and selected from oxygen, dinitrogen, carbon dioxide and mixtures thereof. Some of these nanoparticles may be used in diagnosis (the Gd, Fe ions serving particularly as marking ions) in particular in magnetic resonance imaging (MRI) and in treatment when they are charged with oxygen. Indeed, they then provide oxygen that they deliver to the cells and thus increase the reactivity of cells of solid cancerous tumours to anticancer agents.

According to another aspect of the invention, it relates to a suspension comprising or consisting of the nanoparticles of the invention and of a dispersant selected from water, a buffer consisting of water and of 137 mM/L of NaCl, 2.7 mM/L of KCl, 10 mM/L of Na₂HPO₄, 1.76 mM/L of KH₂PO₄ and having a pH equal to 7.2 (PBS), alcohols and mixtures thereof.

According to another aspect, the present invention relates to a pharmaceutical composition containing as a contrast agent or pharmaceutically active agent nanoparticles according to the invention, possibly suspended in a dispersant.

The amount of nanoparticles is the amount adapted to obtain a therapeutic effect or to obtain a contrast making the diagnosis possible.

The dispersant is selected from pharmaceutically acceptable solvents in particular water, PBS, alcohols and mixtures thereof.

The composition of the invention may be administered orally, mucosally (in particular intranasally) or parenterally.

The present invention also relates to a nanoparticle according to the invention, for the use thereof as a medicinal product in particular for the treatment of hypoxic solid brain tumours and particularly glioblastoma or as a diagnostic agent, in particular as an MRI contrast agent.

The present invention also relates to a combination product comprising or consisting of the pharmaceutical composition of the invention or of the suspension according to the invention and of an anticancer agent, for simultaneous or delayed administration thereof. The nanoparticles according to the invention preferably contain oxygen in the porous network thereof and are optionally suspended in a pharmaceutically acceptable dispersant. The dispersant is preferably selected from water, PBS, and mixtures thereof. The anticancer agent and the pharmaceutical composition of the combination product of the invention may be administered simultaneously or deferred, the anticancer agent being advantageously administered first. The anticancer agent may be selected from Temozolomide® (4-methyl-5-oxo-2,3,4,6,8-pentazabicyclo [4.3.0]nona-2,7,9-triene-9-carboxamide methazolastone), doxorubicin (8S,10S)-10-(4-amino-5-hydroxy-6-methyl-tetrahydro-2H-pyran-2-yloxy)-6,8,11-trihydroxy-8-(2-hydroxyacetyl)-1-methoxy-7,8,9,10-tetrahydrotetracene-5.12-dione), cisplatin (cis-diaminedichloroplatin (II)).

### Definitions

For the purposes of the invention, the term “average size” refers to the average size (in number or intensity) measured by dynamic scattering of the light. The average size is the diameter of the sphere that diffuses at the same rate as that of the particle being measured. The fundamental size distribution obtained by DLS is an intensity distribution. The operating conditions are as defined in the remainder of the application. For the purposes of the invention, the term “nanoparticle” refers to any solid particle with a maximum size of less than 100 nm. It may be a monocrystal or an aggregate of crystals.

The term PBS refers to the saline phosphate buffer which is an aqueous solution of NaCl (0.137 M), KCl (0.0027 M), dibasic sodium phosphate (0.01 M) and monobasic potassium phosphate (0.0018 M). The pH thereof is 7.2.

The term “FAU-type nanoparticle” refers to nanoparticles comprising or consisting of an FAU-type zeolite monocrystal.

The term “FAU-type zeolite” refers to an aluminosilicate that crystallises in a cubical system with centred faces. According to the invention, the terms include X FAU-type zeolites for which the Si/Al molar ratio is greater than or equal to 1 and less than or equal to 1.5 and Y FAU-type zeolites for which the Si/Al molar ratio is greater than or equal to 1.5 and less than or equal to 2.6. Thus, a chemical formula of an FAU-type zeolite nanocrystal is INaₐK_{b} (H₂O)_{c}I(AlₓSi₁₉₂₋ₓO₃₈₄), the Na⁺ and K⁺ ions being present so as to ensure the electrical neutrality of the nanocrystal. Water molecules may be absent from the nanocrystal.

The term “clear suspension” refers to an aqueous suspension having substantially the same refractive index as water, that is to say a refractive index of about 1.333±0.030, measured under standard conditions.

The terms “porous volume” or “porous network” refer to the assembly formed of cavities, channels connecting these cavities together, and pores that connect the channels and cavities outside of the nanoparticle.

The term “pharmaceutically active” refers to an agent that imparts curative or preventive therapeutic properties to the composition containing it and which, if it has adverse side effects, these are not predominant over the curative and/or preventive effects.

The term “pharmaceutically acceptable” refers to any agent that can be ingested, injected, or come into contact with the skin and mucous membranes without causing an adverse effect or without causing an adverse effect of such a magnitude that it disrupts the state of health of the patient.

The term “composition” refers to any homogeneous or inhomogeneous mixture of at least two compounds that may be in a different physical state; the term composition thus includes particularly solutions, suspensions and dispersions.

### BRIEF DESCRIPTION OF THE FIGURES

The features and advantages of the invention will become apparent upon reading the following description based on the figures, wherein:

FIG. 1 shows the size distribution of the Na-FAU-PBS nanoparticles of the invention, the nanoparticles are dispersed in a PBS solution;

FIG. 2A shows images obtained by scanning electron microscope of Na-FAU-PBS and Gd-FAU-PBS-type nanoparticles (nanocrystals) with a scale of 500 nm and FIG. 2B shows images obtained by scanning electron microscope of Na-FAU-PBS-type nanoparticles (nanocrystals) with a scale of 100 nm and 40 nm;

FIG. 3 shows the amount of dinitrogen adsorbed as a function of the relative pressure obtained under the operating conditions described below for the Na-FAU-PBS-type nanoparticles;

FIG. 4 shows a comparison of the viability of the U87-MG cells exposed to two colloidal suspensions of nanoparticles containing respectively 10 μg/mL and 100 μg/mL of Na-FAU-Water or Gd-FAU-Water nanoparticles with that obtained for the same concentrations of Na-FAU-PBS and Gd-FAU-PBS nanoparticles;

FIG. 5 shows on the left the percentage of hemolysis as a function of the dose of Na-FAU- and Gd-FAU-type nanoparticles (in μg/mL) synthesised with water or PBS;

FIG. 6A shows the change in oxygen concentration in water obtained by placing in contact with Na-FAU-Water and Gd-FAU-Water particles, previously charged with O₂ and FIG. 6B the change in oxygen concentration in the PBS obtained by placing in contact with Na-FAU-PBS and Gd-FAU-PBS particles of the invention and previously charged with O₂;

### EXAMPLES

### Synthesis and Characterisation of Nanoparticles

The raw materials used in the following examples are listed below:
Sodium hydroxide (Sigma Aldrich, 98%)
Potassium hydroxide (Sigma Aldrich, 90%)
Aluminium hydroxide (type A2100, Alfa Aesar, 76.5% by mass Al(OH)₃, 23.5% by mass H₂O)
Aluminium powder (325 mesh, Alfa Aesar 99.5%)
Colloidal silica (SiO₂ (Ludox HS-30, 30 wt. % SiO₂, pH=9.5-10.2 Sigma Aldrich)
Colloidal silica (SiO₂ (Ludox SM-30, 30 wt. % SiO₂, pH=9.7-10.3 Sigma Aldrich)
Gadolinium nitrate Gd(NO₃)₃, 6H₂O (Aldrich, 99.9%)
Saline phosphate buffer (PBS) (Sigma Aldrich, pH=7.15-7.25)

These starting reagents were used as received from the manufacturers, without further purification.

### Methods of Analysis Used and Operating Conditions

The various zeolite materials obtained in the examples were characterised on various size scales.

### Scanning and Transmission Electron Microscopy (SEM/TEM) Imaging:

The morphology of the nanoparticles was examined by scanning electron microscopy (SEM) using a Tescan Mira I LMH device at a voltage of 20 kV.

Transmission electron microscopy (TEM): Colloidal suspensions are sonicated for 15 minutes, then 2 to 3 drops of fine particle suspensions are dried on 300 mesh copper electron microscope grids covered with a carbon film. The images were obtained using a Tecnai G2 30 FEI microscope (Vacc=300 kV, LaB6).

### Dynamic Light Scattering (DLS) Analysis:

The hydrodynamic diameters of the zeolite material in the various suspensions were determined with a Malvern Zetasizer Nano device. The surface charge of the samples was determined by measuring the zeta potential of the suspensions. The analyses were carried out on samples after purification with a solid concentration of 1% by weight dispersed in a PBS solution at pH=7.2. The backscattering geometry (scattering angle 173°, HeNe laser with an output power of 3 mW at a wavelength of 632.8 nm) makes measurements possible at high sample concentration, as full penetration of incident light through the sample is not required.

### Chemical Analysis by ICP-MS (Inductively Coupled Plasma Mass Spectrometry)

The chemical composition of the zeolite samples was characterised by inductively coupled plasma mass spectrometry using Agilent Technologies 7900 equipment. The samples were prepared according to the following procedure: (i) 50 mg of zeolite sample were dissolved in 3 mL of hydrofluoric acid (HF) (Sigma-Aldrich 40-45%), (ii) a solution of 0.5 mL (HNO₃/HCl equals 1:3 v/v) was added then the mixture was heated at 110° C. for 1 h in a polytetrafluoroethylene (PTFE) bottle (100 mL), and then (iii) 96.5 mL of distilled water and 2 g of boric acid (H₃BO₃) were added. The solution obtained was stirred overnight to facilitate the dissolution of the boric acid. Finally, 10 mL of the suspension were diluted 10 times with distilled water before analysis.

### Example 1: Synthesis of FAU-Type Nanocrystals in a PBS Solution (Na-FAU-PBS)

The colloidal suspension of FAU was synthesised by hydrothermal means from the molar composition cited with reference to the first embodiment of the method of the invention.

A solution A of aluminium powder is prepared by dissolving 2.3 g of NaOH in 3 g of PBS followed by a slow addition of 0.29 g of aluminium powder.

A silicate solution B is prepared by mixing 10 g of colloidal silica (LUDOX® HS-30) with 1.1 g of NaOH and 1 g of PBS. This turbid solution (suspension B) was heated for 6-7 min in an oven at 100° C. to be transformed into a clear solution. Solution B was subsequently added drop by drop to solution A at a temperature of 0-3° C. and rapid stirring, then the mixture was aged for 24 h at room temperature.

The initial molar composition of the mixture is as follows:

8.3Na2O:1.1Al₂O₃:10SiO₂:55.6 (PBS)

The mixture obtained is dehydrated by lyophilisation and then crystallised for 25 h in an oven at 50° C. The suspension is purified by dialysis membrane (Visking Dialysis Tubing, Code No.-DTV.12000.08 Size No. 8 (25.4 mm, 30 m), Molecular weight 12-14,000 Daltons), until a pH of 7.2 is reached. 60-75 mL (1.6 to 1.9 g) of a colloidal suspension containing 2.5% by mass of Na-FAU-type zeolite nanocrystals in PBS is obtained. The nanoparticles include in the porous network thereof ions of the PBS and as an alkaline ion interposed in the network, Na⁺ and K⁺ ions. The final suspensions were subjected to sonication for 2 h.

The crystal yield ranges from 53 to 63% (the crystal yield is defined as the mass of product obtained X 100/mass of SiO₂ contained in solution B.

Chemical formula of the Na-FAU-PBS zeolite nanocrystal obtained by ICP:

|Na_{69.79}K_{6.06} (H₂O)_{211.85}|(Si_{123.09}Al_{66.59}O₃₈₄)

The formula between brackets corresponds to the crystal lattice, the number of moles of compensation ions and the number of moles of water are shown between the bars.

### Example 2: Synthesis of Colloidal Suspensions of Nanoparticles According to the Invention

A given amount of each of the types of nanoparticles of Example 1 was dispersed in 10 mL of PBS under sonication for 2 h. Colloidal solutions containing respectively 1%, 2%, 3% and 4% by mass of Na-FAU-PBS-type nanoparticles dispersed in PBS are obtained.

These nanoparticles are stable in colloidal suspension at room temperature for at least one week.

### Example 3: Synthesis of Zeolite Nanoparticles Charged with Metal Ions

5 mL of each of the two colloidal suspensions obtained in Example 2, each containing 2.5% by mass of nanoparticles were added separately to 25 mL of a gadolinium nitrate solution (III) hexahydrate (Gd(NO₃)₃·6H₂O) with a concentration of 0.03 mM). The suspension is subsequently left under stirring at room temperature for 1 h. This ion exchange process was carried out twice. The final suspension was subsequently washed 3 times with PBS to finally obtain a suspension of Gd-FAU-PBS zeolite in PBS and therefore at pH=7.2.

### Example 4: Incorporation of Dioxygen and/or CO₂ into the Porous Network of Nanoparticles

Each of the colloidal aqueous suspensions obtained in Example 2 or Example 3 was subsequently splashed with O₂, CO₂ or carbogen at a flow rate of 800 mL/min for 30 minutes.

Nanoparticles are thus obtained the porous network of which is at least partially filled with O₂ and/or CO₂.

### Example 5: Morphological Characterisation of the Nanoparticles Obtained in Examples 1, 2 and 4

Scanning/transmission electron microscopy (SEM)/(TEM) was used to determine the morphology and size of synthesised Na-FAU-PBS and Gd-FAU-PBS nanoparticles

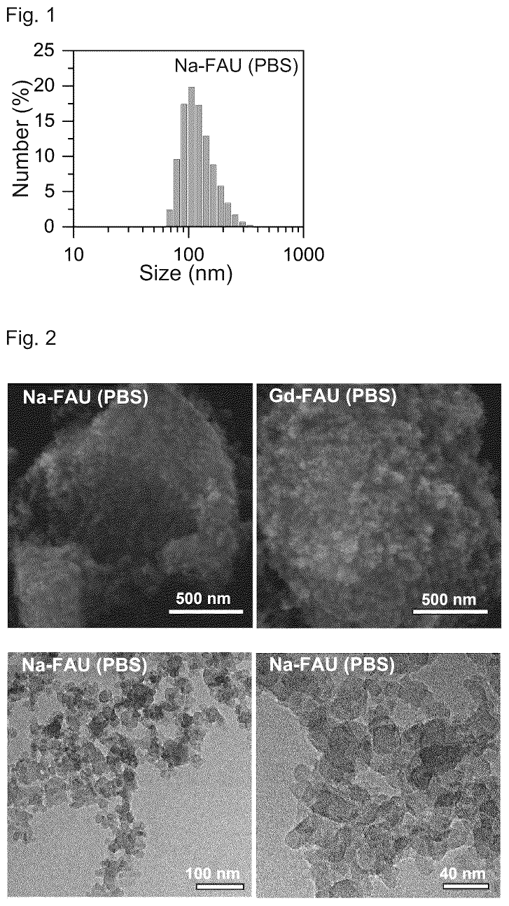

The curve giving the size distribution of the dispersed nanoparticles in the PBS is shown in FIG. 1 for Na-FAU-PBS nanoparticles.

For Na-FAU-PBS-type nanoparticles, the hydrodynamic size of the aggregates of nanoparticles in a PBS solution measured by DLS is between 50-200 nm. It is observed that this is a mono-dispersed population.

The scanning electron microscopy images of Na-FAU-PBS or Gd-FAU-PBS zeolite nanoparticles (see FIG. 2A) show nanoparticles with a quasi-spherical shape with a homogeneous size distribution and the transmission electron microscopy TEM images (see FIG. 2B) show nanoparticles (nanocrystals) having an average size of 30 nm. In addition, the high resolution transmission electron microscopy images show that nanoparticles are highly crystalline. These are nanoparticles each formed from a single nanocrystal.

### Example 6: Chemical Analysis of Na-FAU Nanoparticles

The chemical formula of the zeolite material of the Na-FAU-PBS and Gd-FAU-PBS nanoparticles is as follows (determined by ICP):

|Na_{69.79}K_{6.06} (H₂O)_{211.85}|(Si_{123.09}Al_{66.59}O₃₈₄) (II).

|Na_{63.92}K_{2.33}Gd_{2.44} (H₂O)_{165.49}|(Si_{123.26}Al_{67.26}O₃₈₄) (II).

The formula between brackets corresponds to the tetrahedral crystal lattice, the number of moles of compensation cations and the number of moles of water are shown between the bars.

### Example 7: Determination of Porosity by Nitrogen Adsorption/Desorption

The porosity of the Na-FAU-PBS and Gd-FAU-PBS zeolites synthesised in the aforementioned examples was characterised by measurements of gaseous nitrogen adsorption/desorption.

The textural properties of the nanoparticles obtained in Examples 1, 2 and 4 were determined by the nitrogen adsorption/desorption isotherms measured at a temperature of −196° C. using a “Micromeritics 3Flex” surface characterisation device. All of the samples were degassed at 250° C. under vacuum overnight before analysis. The outer surface of the zeolite nanoparticles and the volume of the micropores of the zeolite nanoparticles were evaluated by the t-plot method.

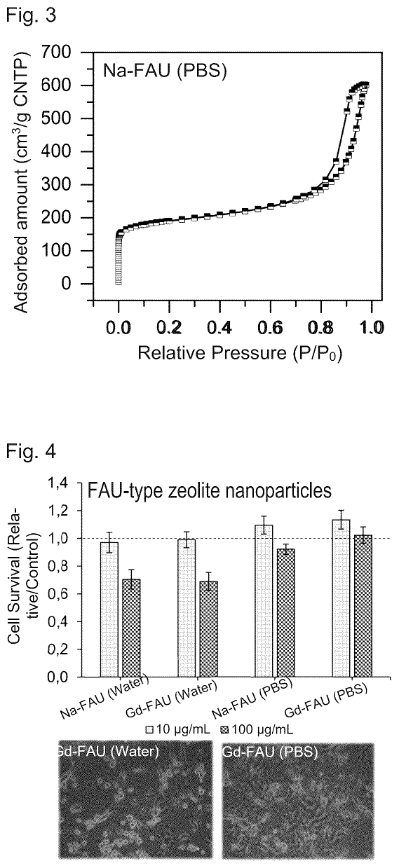

The nitrogen adsorption/desorption measurements for the two zeolites resulted in hybrid isotherms of type I and IV according to the IUPAC classification (see FIG. 3). The measurement of the specific surface area S_{BET}, the total porous volume and the volume of the micropores of the zeolite nanoparticles are grouped in Table 1 below. A micropore is defined as a pore smaller than 0.08 mm in size.

TABLE 1

| | | Total pore | Micropore |
|---|---|---|---|
| | S_{BET} | volume | volume |
| Nanoparticle | (m²/g) | (cm³/g) | (cm³/g) |
| Na-FAU-PBS | 600 | 0.93 | 0.19 |
| Gd-FAU-PBS | 428 | 0.93 | 0.14 |

Table 2 below groups the chemical composition of the Na-FAU-PBS and Gd-FAU-PBS nanoparticles of the invention.

TABLE 2

| | Chemical composition (% by mass) | | | | | |
|---|---|---|---|---|---|---|
| Samples | K | Na | Si | Al | Gd | Si/Al |
| Na-FAU-PBS | 1.23 | 8.33 | 17.96 | 9.33 | — | 1.85 |
| Gd-FAU-PBS | 0.48 | 7.95 | 18.36 | 9.61 | 2.03 | 1.84 |

The pharmacological properties of the nanoparticles of the invention were studied. The results are shown in the following examples.

### Example 8: Human Cell Line Toxicity Test

The cells used come from a human glioblastoma cell line, U87-MG purchased from American Type Culture Collection (ATCC). The cells were cultured in DMEM 1 g/L glucose (Sigma-Aldrich) supplemented with 10% foetal calf serum (Eurobio), 2 mM glutamine (Sigma-Aldrich) and penicillin (100 U/mL)/streptomycin (100 g/mL) (Sigma-Aldrich). The cells were kept in culture at 37° C. with 5% of CO₂ and 95% of humidity. The cell viability was evaluated 72 h after exposure to zeolite nanoparticles with the WST-1 assay (Roche) according to the instructions of the manufacturer.

The results can be seen in FIG. 4 5. This figure also shows the percentage of cell viability with exposure to nanoparticles synthesised in water instead of PBS. It is observed that for the Na-FAU-PBS, Gd-FAU-PBS nanoparticles of the invention, the percentage of viable cells is higher than for the nanoparticles synthesised from the same mixture but with water instead of PBS (Na-FAU-Water, Gd-FAU-Water).

### Example 9: Viability Test of Red Blood Cells

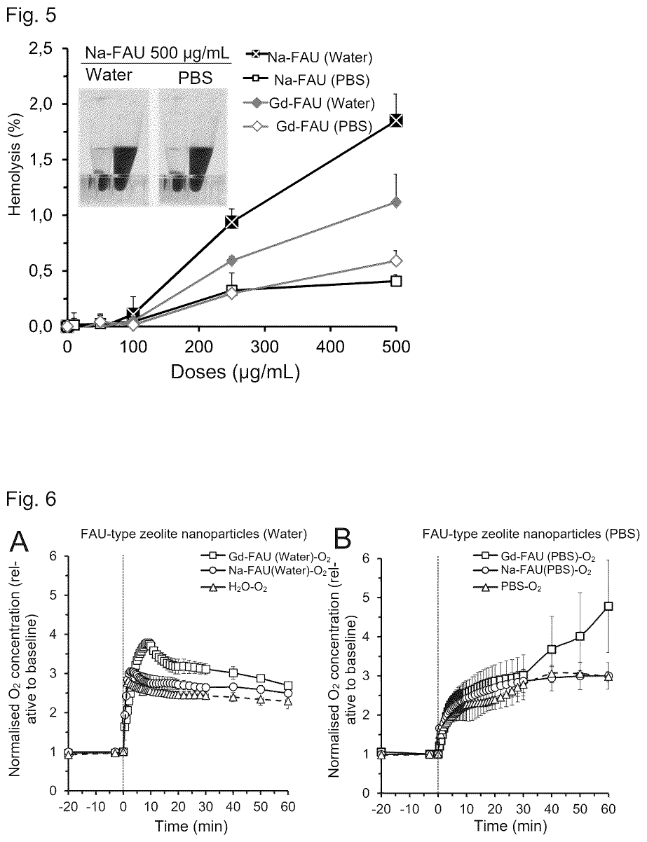

To confirm the absence of toxicity of the nanoparticles of the invention, red blood cells were exposed to increasing concentrations of nanoparticles according to the invention (from Examples 1, 2 and 4). The results can be seen in FIG. 5. It is thus observed that for nanoparticles synthesised in water, the maximum percentage of hemolysis is 1.85% for Na-FAU-Water nanoparticles and 1.19% for Gd-FAU-Water nanoparticles. This percentage decreases to 0.408% and 0.590% for Na-FAU-PBS and Gd-FAU-PBS particles, respectively.

Moreover, in the presence of a large amount of nanoparticles (500 μg/mL), the highest percentage of hemolysis obtained for the Gd-FAU-Water nanoparticles is 2%. It drops to 1% for Gd-FAU-PBS nanoparticles.

These results confirm that the use of PBS during the synthesis of nanoparticles decreases the toxicity thereof.

### Example 10: Study of Oxygen Release by Zeolite Nanoparticles in Aqueous and Hypoxic Conditions

A hypoxia chamber (IN VIVO2,500™, 3M) was used to obtain a stable and precisely controlled gas composition of the atmosphere with an accuracy of 0.1% O₂ by adapting the amount of N₂. The PBS solution (Sigma-Aldrich) was equilibrated with the gas mixture contained in the hypoxic chamber for 1 h before the experiment. A closed tube containing 12 mL of equilibrated PBS at 37° C. and a dissolved oxygen sensor (SevenGo (Duo) Pro™/OptiOx™, Mettler Toledo) were used inside the hypoxic chamber. Before the experiment, the baseline was established by measuring the oxygen content of the PBS for 30 min.

The nanoparticles of the examples of the various types and previously charged with oxygen were subsequently added to the system and the oxygen dissolved in the PBS solution was measured continuously for 1 h. The results can be seen in FIGS. 6A and 6B. It is observed that the Gd-FAU-PBS nanoparticles of the invention previously charged with oxygen have a better dioxygen release compared to the nanoparticles prepared in water (Gd-FAU-Water). In addition, the release kinetics are also increased, which is interesting for in vivo use. The oxygen release is accelerated after 30 min, which is of considerable interest for in vivo use, as the particle has time to reach the brain and deliver oxygen there. Regarding Na-FAU-PBS nanoparticles, it is observed that the dioxygen release kinetics is slower compared to Na-FAU-Water.

1. A method for synthesising nanoparticles consisting of or comprising at least one zeolite nanocrystal according to which:
a first composition/solution 1 containing an aluminium source and a source of an ion of an alkali metal M, in particular Na, is prepared;
a second composition/solution 2 comprising a silicon source and a source of an ion of an alkali metal M in particular Na, is prepared, said compositions/solutions 1 and 2 being free of any organic structuring agent;
the compositions/solutions 1 and 2 are mixed and the mixture is left under stirring,
the mixture is crystallised at a temperature greater than or equal to 50° C.; and
said nanoparticles thus formed are optionally separated,
wherein said first composition/solution 1 and said second composition/solution 2 are each consisting of said source and of an aqueous buffer consisting of water and of 137 mM/L of NaCl, 2.7 mM/L of KCl, 10 mM/L of Na₂HPO₄, 1.76 mM/L of KH₂PO₄ and having a pH equal to 7.2 (PBS) and in that said mixture of said compositions/suspensions 1 and 2 has the following molar composition (I):
X1M₂O:YAl₂O₃:10SiO₂:Z PBS (I)
wherein 2.8≤X1≤8.8,
$$0.3 \leq Y \leq 1.5 ,$$
$$40 \leq Z \leq 30 \; 0 .$$

2. The method according to claim 1, wherein M=Na, in that said aluminium source is aluminium powder, in that before crystallising said mixture of said compositions/solutions 1 and 2, it is dehydrated under vacuum and in that the remaining solid fraction is subsequently crystallised at a temperature greater than or equal to 30° C. and less than or equal to 100° C. and in particular equal to 50° C. for a duration equal to or greater than 6 h and equal to or less than 30 h and particularly equal to 25 h.

3. The method according to claim 1, wherein M=Na and in that 5≤x1≤8.8, 0.3≤y≤1.5 and 40≤z≤200 and in particular x1=8.3, y=1.1 and z=55.6.

4. The method according to claim 1, wherein said nanoparticles are separated by membrane filtration.

5. A nanoparticle consisting of or comprising at least one zeolite nanocrystal and obtainable according to the method of claim 1, wherein said zeolite is an FAU-type aluminosilicate and in that it contains Na⁺, K⁺, HPO₄²⁻, H₃O⁺, H₂PO₄⁻ and Cl⁻ ions.

6. The nanoparticle according to claim 5, wherein said nanocrystal has the following formula (II):

|Na_{69.79}K_{6.06} (H₂O)_{211.85}| (Si_{123.09}Al_{66.59}O₃₈₄) (II) or the following formula

|Na_{63.92}K_{2.33}Gd_{2.44} (H₂O)_{165.49}|(Si_{123.26}Al_{67.26}O₃₈₄)

7. The nanoparticle according to claim 5, wherein it has a total porous volume greater than or equal to 0.4 cm³/g and less than or equal to 1.5 cm³/g and/or a specific surface area S_{BET} greater than or equal to 200 m²/g and less than or equal to 1,000 m²/g.

8. The nanoparticle according to claim 5, wherein it further contains in the crystal lattice thereof cations of a metal selected from Fe, Gd, Cu and Ce and in particular in an amount greater than or equal to 0.2% by weight and less than or equal to 5% by weight and/or a gas contained in the porous volume thereof and selected from oxygen, dinitrogen, carbon dioxide and mixtures thereof.

9. A suspension comprising or consisting of nanoparticles according to claim 1 and of a dispersant selected from water, a buffer consisting of water and of 137 mM/L of NaCl, 2.7 mM/L of KCl, 10 mM/L of Na₂HPO₄, 1.76 mM/L of KH₂PO₄ and having a pH equal to 7.2, alcohols and mixtures thereof.

10. A pharmaceutical composition containing as a contrast agent or pharmaceutically active agent nanoparticles according to claim 5, optionally suspended in a dispersant or the suspension according to claim 9.

11. The pharmaceutical composition according to claim 10, wherein said dispersant is selected from water, a buffer consisting of water and of 137 mM/L of NaCl, 2.7 mM/L of KCl, 10 mM/L of Na₂HPO₄, 1.76 mM/L of KH₂PO₄ and having a pH equal to 7.2, alcohols and mixtures thereof.

12. A combination product comprising the pharmaceutical composition according to claim 10 and an anticancer agent for simultaneous or delayed administration thereof.

13. The nanoparticle according to any claim 5 for the use thereof as a medicinal product, particularly for the treatment of solid tumours in particular of the brain, in particular glioblastoma, or for the use thereof
as a contrast agent, particularly in magnetic resonance imaging (MRI).

14. The suspension according to claim 9, particularly in PBS, for the use thereof as a medicinal product, particularly for the treatment of solid tumours in particular of the brain, in particular glioblastoma.

15. The suspension according to claim 9, particularly in PBS, for the use thereof as a contrast agent, in particular in magnetic resonance imaging.

16. A combination product comprising the suspension of claim 9 and an anticancer agent for simultaneous or delayed administration thereof.
